(19) United States
(12) Patent Application Publication
(10) Pub. No.: US 2021/0381902 A1
(43) Pub. Date: Dec. 9, 2021

(54) HOLDER FOR A TEMPORAL THERMOMETER

(71) Applicant: Dynabrade, Inc., Clarence, NY (US)

(72) Inventor: John Thomas Swaine, Buffalo, NY (US)

(21) Appl. No.: 16/896,800

(22) Filed: Jun. 9, 2020

(51) Int. Cl. G01K 1/14; A61B 5/01; A61B 5/00; F16M 13/02; G05G 1/46; A47G 1/04; G05G 7/00; F16M 11/22; F16C 1/12

(52) U.S. Cl. CPC G01K 1/14; A61B 5/01; A61B 5/742; F16M 13/02; F16C 1/12; A47G 1/04; G05G 7/00; F16M 11/22; G05G 1/46

(57) ABSTRACT

A holder for a temporal thermometer, having a bracket arranged to hold the thermometer, a lever operatively arranged to depress a button on the thermometer to take a temperature reading, a foot pedal, a cable linking the foot pedal to the lever to activate the thermometer, and a duplex mirror assembly operatively arranged to display the temperature reading.

### FIELD OF THE INVENTION

The present invention generally relates to a bracket and pedal system that holds and operates a temporal thermometer without direct hand contact.

### BACKGROUND OF THE INVENTION

During early 2020, SARS-CoV-2, also known as the Coronavirus or COVID-19 spread worldwide, requiring countries, states, and individual counties to initiate lockdown procedures to flatten the curve of the pandemic spread. As companies reopened, many businesses began providing initial testing to safely gauge whether employees were healthy and could return to work. They also implemented procedures to test employees as they entered the workplace to ensure that they were symptom-free. Symptoms of a positive COVID-19 patient include a fever. Therefore, taking the temperature of a worker prior to entering the workplace, is a cost-effective measure to prevent increased spread of the virus. Other precautions that were established to help curb the spread of the virus included social distancing and curbside pick-up to encourage less physical contact.

A previous solution to screen employees for COVID-19 has been the installation of thermal camera technology that can detect heat emitted from an individual's body. Major companies such as Amazon, Tyson Foods, and Intel have used these thermal cameras. The camera systems are known in the art and were widely used in Asian airports after the SARS epidemic in 2003. However, these sophisticated camera systems are expensive (between $5,000 and $20,000), and unaffordable by many smaller companies.

One inexpensive option for smaller companies is the use of an infrared temporal thermometer. Temporal artery thermometers—often sold as forehead thermometers—use an infrared scanner to measure the temperature of the temporal artery in the forehead. An advantage of this type of thermometer, in addition to their relatively low cost, is that it can record a person's temperature quickly and is reasonably accurate. In fact, some research suggests that temporal artery thermometers are the most accurate alternative to rectal digital thermometers for taking a child's temperature. A disadvantage, however, is that, in use, someone must hold and operate the thermometer to record the temperature of another. Obviously, it is an added expense to pay someone to record the temperatures of co-workers and visitors.

Thus, there has been a long-felt need for a low-cost apparatus for measuring temperature of employees entering a workplace with a temporal thermometer, while avoiding the need for a person to manually hold the thermometer and take readings, and also avoiding contact with the thermometer by the person whose temperature is being recorded.

### SUMMARY OF THE INVENTION

The present invention comprises a holder for a temporal thermometer, having a bracket arranged to hold the thermometer, a lever operatively arranged to depress a button on the thermometer to take a temperature reading, a foot pedal, a cable linking the foot pedal to the lever to activate the thermometer, and a duplex mirror assembly operatively arranged to display the temperature reading.

In an alternative embodiment, the present invention comprises a holder for a temporal thermometer, having a bracket arranged to hold the thermometer, a lever operatively arranged to depress a button on the thermometer to take a temperature reading, a foot pedal, a cable linking the foot pedal to the lever to activate the thermometer, a conduit arranged to cover the cable, and a duplex mirror assembly operatively arranged to display the temperature reading.

In another alternative embodiment, the present invention comprises a holder for a temporal thermometer, having a bracket arranged to hold the thermometer, a lever operatively arranged to depress a button on the thermometer to take a temperature reading, a foot pedal, a cable linking the foot pedal to the lever to activate the thermometer, a flexible conduit arrange to cover the cable, and a duplex mirror assembly operatively arranged to display the temperature reading.

A general object of the invention is to provide an inexpensive holder for a temporal thermometer for quick and easy contactless measurement of employee temperatures in a workplace.

A secondary object of the invention is to provide a holder for a temporal thermometer that may be operated by the person whose temperature is to be recorded without need of another employee to operate the thermometer.

A further object of the invention is to provide a foot operated device for activating a temporal thermometer.

These and other objects, features and advantages of the invention will become readily apparent upon a reading of the following detailed description in view of the drawings and appended claims.

### BRIEF DESCRIPTION OF THE DRAWINGS

The nature and mode of the operation of the present invention will now be more fully described in the following detailed description of the invention taken with the accompanying drawing figures, in which:

FIG. 1 is a perspective view of a first embodiment of the present invention;

FIG. 2 is a perspective view of a second embodiment of the present invention;

FIG. 3A is a left side view of the invention shown in FIG. 1;

FIG. 3B is a fragmentary left side view of the thermometer holding bracket and duplex mirror assembly, common to both embodiments of the invention, shown with a thermometer held in the bracket;

FIG. 3C is a fragmentary left side view of the foot pedal, common to both embodiments of the invention;

FIG. 4A is a right side view of the invention shown in FIG. 1;

FIG. 4B is a fragmentary right side view of the thermometer holding bracket and duplex mirror assembly, common to both embodiments of the invention, shown with a thermometer held in the bracket;

FIG. 4C is a fragmentary right side view of the foot pedal, common to both embodiments of the invention;

FIG. 5A is a front view of the first embodiment of the invention shown in FIG. 1;

FIG. 5B is a fragmentary front view of the thermometer holding bracket and duplex mirror assembly, common to both embodiments of the invention, shown with a thermometer held in the bracket;

FIG. 5C is a fragmentary front view of the foot pedal, common to both embodiments of the invention;

FIG. 6A is a rear view of the first embodiment of the invention shown in FIG. 1;

FIG. 6B is a fragmentary rear view of the thermometer holding bracket and duplex mirror assembly, common to both embodiments of the invention, shown without a thermometer held in the bracket;

FIG. 6C is a fragmentary rear view of the foot pedal common to both embodiments of the invention;

FIG. 7 is a top perspective view of the thermometer holding bracket and duplex mirror assembly, common to both embodiments of the invention, shown without a thermometer held in the bracket;

FIG. 8 is a top-right perspective view of the thermometer holding bracket and duplex mirror assembly, common to both embodiments of the invention, shown without a thermometer held in the bracket;

FIG. 9 is a bottom-left perspective view of the thermometer holding bracket and duplex mirror assembly, common to both embodiments of the invention, shown without a thermometer held in the bracket;

FIG. 10 is a bottom-right perspective view of the thermometer holding bracket and duplex mirror assembly, common to both embodiments of the invention, shown without a thermometer held in the bracket;

FIG. 11 is an exploded view of the thermometer holding bracket and duplex mirror assembly, common to both embodiments of the invention;

FIG. 12 is an exploded view of the pedal assembly; and,

FIG. 13 is a perspective view of a flexible conduit embodiment.

### DETAILED DESCRIPTION OF EMBODIMENTS OF THE INVENTION

The terminology used herein is for the purpose of describing particular embodiments only and is not intended to be limiting of the invention.

As used herein, the term “and/or” includes any and all combinations of one or more of the associated listed items. As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion.

Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art, that the present invention may be practiced without these specific details.

The present disclosure is to be considered as an exemplification of the invention, and is not intended to limit the invention to the specific embodiments illustrated by the figures or description below.

It should be appreciated that two embodiments of the invention are described and claimed herein. In a first embodiment, a rigid conduit connects the pedal assembly to the thermometer holder, which conduit carries and protects the pedal cable. The second embodiment does not include this conduit and the pedal cable is exposed. This embodiment is preferred for shipping and it permits a more compact package for the invention.

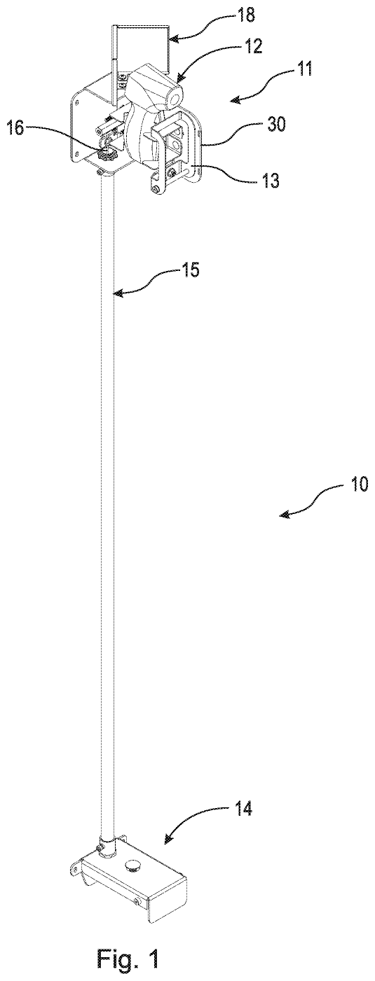

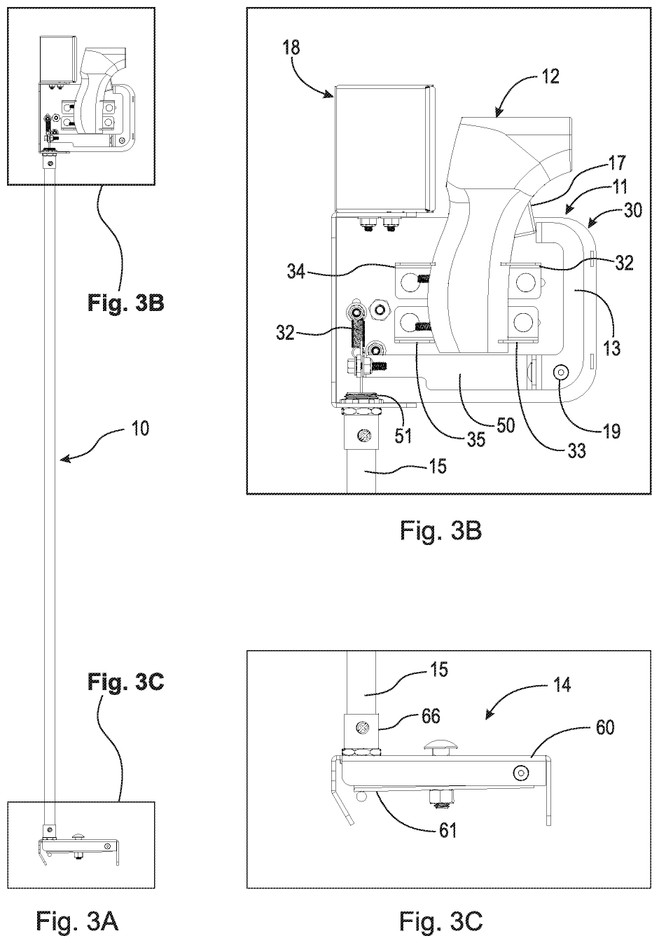

The present invention will now be described by referencing the appended figures representing a first and second embodiment. FIG. 1 is a perspective view of a first embodiment of the present invention, showing temporal thermometer holder 10. Temporal thermometer holder 10 comprises holder assembly 11, thermometer 12, trigger 13, pedal assembly 14, conduit 15, cable 16, thermometer button 17, and duplex mirror 18. Holder assembly 11 secures temporal thermometer 12 in a position so that trigger 13 may engage and activate thermometer 12 to take a temperature reading. Pedal assembly 14 and trigger 13 are connected by way of cable 16 so that when pedal assembly 14 is depressed, cable 16 engages trigger 13 to depress thermometer button 17 to take a temperature reading. Thermometer button 17 is shown in FIG. 3B and discussed infra.

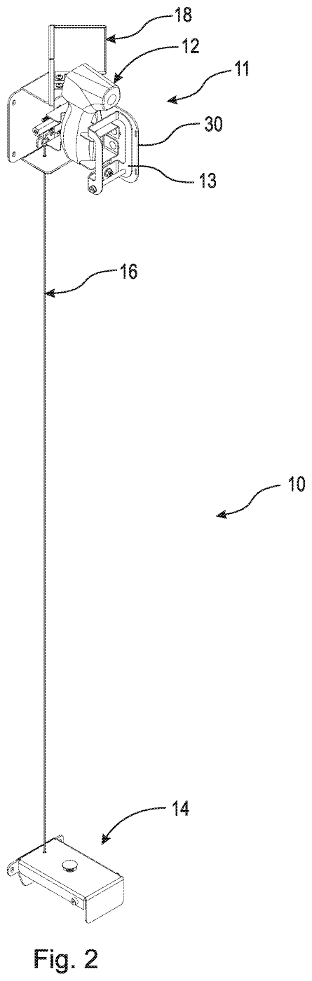

FIG. 2 shows a perspective view of a second embodiment of temporal thermometer holder 10 comprising, bracket 11, thermometer 12, trigger 13, pedal assembly 14, cable 16, and duplex mirror 18. This second embodiment is virtually identical to the first embodiment, except it does not include the conduit and the pedal cable is therefore exposed. This embodiment is preferred for shipping as it permits a more compact package for the invention.

FIG. 3A is a left side view of temporal thermometer holder 10 with the temporal thermometer positioned in the holder for use as shown in FIG. 1. The following description should be read in view of FIGS. 3A-3C.

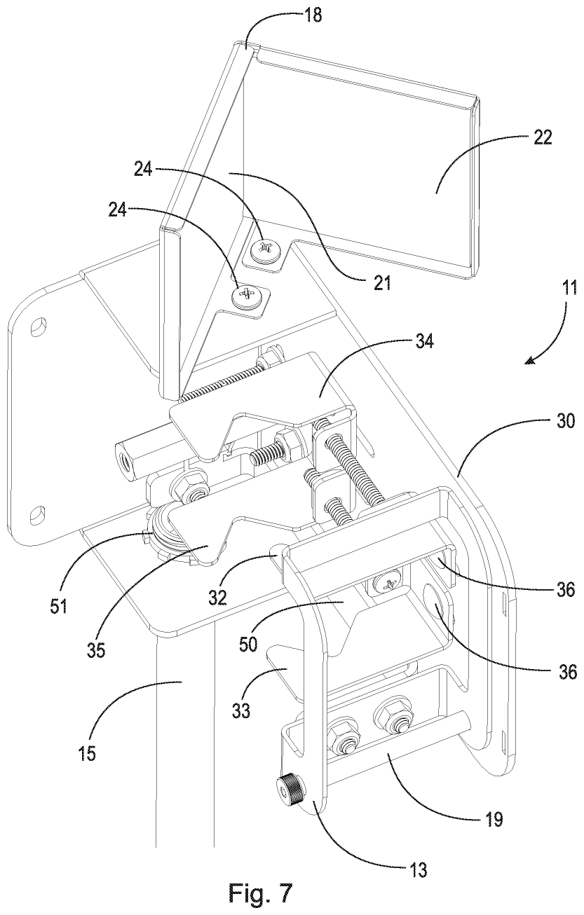

FIG. 3B is an enlarged view of the part of the invention shown in the rectangle labeled FIG. 3B in FIG. 3A. FIG. 3B is a fragmentary left side view of bracket 11 and duplex mirror assembly 18, common to both embodiments of the invention, shown with temporal thermometer 12 held in holder assembly 11. Holder assembly 11 comprises bracket 30 which further comprises trigger 13, trigger bar 50, and duplex mirror 18. Temporal thermometer 12 is secured to bracket 30 by way of four (4) clips. Upper front clip 32, lower front clip 33, upper rear clip 34, and lower rear clip 35 all snuggly secure temporal thermometer 12 within bracket 30. The four (4) clips will be explained in further detail in view of FIG. 7, infra. Trigger bar 50 is rotatably secured to bracket 30 and threadably secured to trigger 13. Trigger bar 50 is operatively arranged to engage trigger 13 to depress thermometer button 17 on temporal thermometer 12 to take a temperature. Trigger bar 50 is secured to the top end of cable 16. Top end of cable 16 is secured to the distal end of bracket 30 by way of conduit electrical metallic (EMT) fitting 51 that is secured to the top end of conduit 15. Extension spring 52 is bolted to bracket 30 and trigger bar 50.

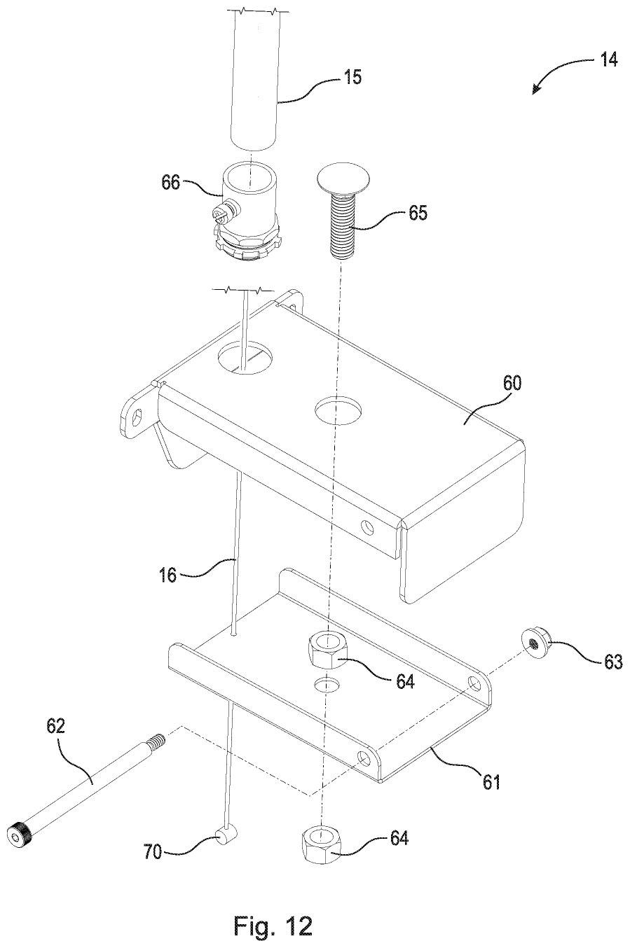

FIG. 3C is an enlarged view of the part of the invention shown in the rectangle labeled FIG. 3C in FIG. 3A. FIG. 3C is a fragmentary left side view of the foot pedal, common to both embodiments of the invention, discussed below and in greater detail supra. Pedal assembly 14 comprises, foot switch base 60, foot switch lever 61, and pedal EMT fitting 66 fixedly securing conduit 15 to foot switch base 60. Cable 16 is secured to foot switch lever 61, shown in FIG. 12 and discussed infra.

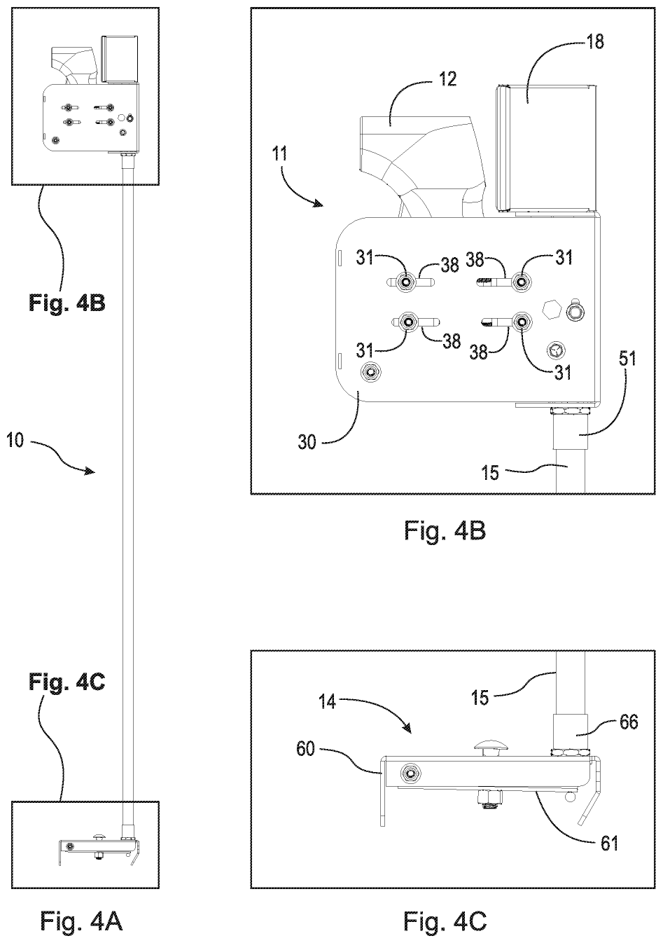

FIG. 4A is a right side view of temporal thermometer holder 10 shown in FIG. 1. The following description should be read in view of FIGS. 4A-4C.

FIG. 4B is an enlarged view of the part of the invention shown in the rectangle labeled FIG. 4B in FIG. 4A. FIG. 4B is a fragmentary right side view of holder assembly 11, common to both embodiments of the invention. Bracket 30 is secured to conduit 15 by way of conduit EMT fitting 51. Bracket 30 further comprises duplex mirror 18 bolted to top surface of bracket 30, discussed further supra. Bracket 30 comprises four (4) clip bolt grooves 38 that are operatively arranged to accept four (4) clip carriage bolts (See, FIG. 7-10 discussed, infra) and their respective flange lock nuts 31 to secure four (4) clips (See, FIG. 3A-3C discussed, supra). Clip bolt grooves 38 are operatively arranged to slidably adjust four (4) clips to accommodate different sized temporal thermometer handles.

FIG. 4C is an enlarged view of the part of the invention shown in the rectangle labeled FIG. 4C in FIG. 4A. FIG. 4C is a fragmentary right side view of the foot pedal, common to both embodiments of the invention showing pedal assembly 14 and conduit 15. Pedal assembly 14 comprises foot switch base 60, foot switch lever 61, and pedal EMT fitting 66 fixedly securing conduit 15 to foot switch base 60.

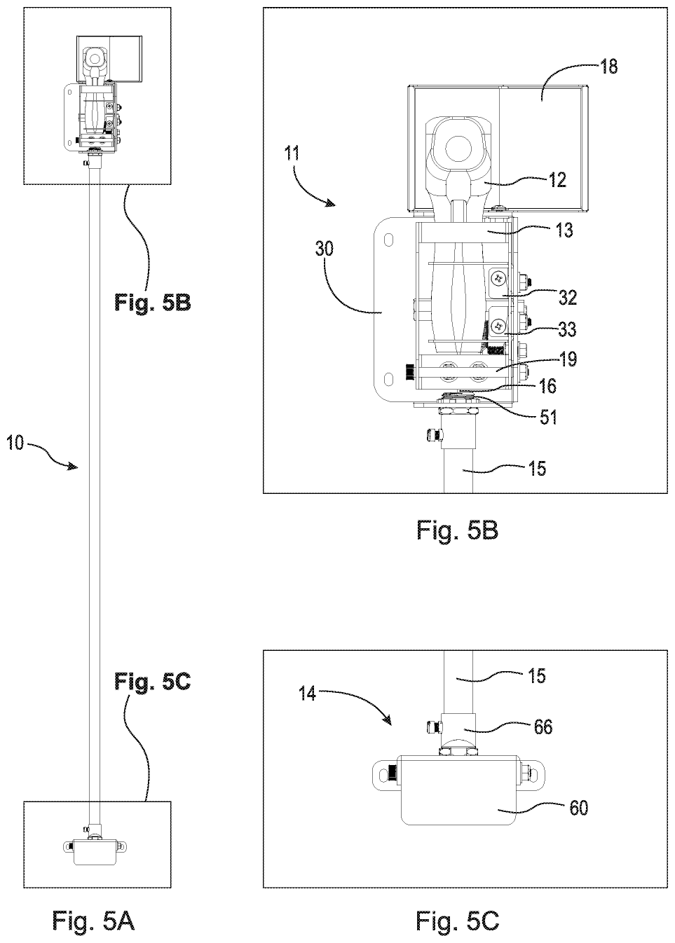

FIG. 5A is a front-side view of temporal thermometer holder 10 with the temporal thermometer positioned in the holder. The following description should be read in view of FIGS. 5A-5C.

FIG. 5B is an enlarged view of the part of the invention shown in the rectangle labeled FIG. 5B in FIG. 5A. FIG. 5B is a partial front-side view of holder assembly 11 with temporal thermometer 12. Bracket 30 is secured to conduit 15 by conduit EMT fitting 51. Duplex mirror 18 is bolted to the top surface of bracket 30 operatively arranged to reflect temporal thermometer 12 temperature to a user. Temporal thermometer 12 is held by four (4) clips bolted to the side of bracket 30. Upper front clip 32 and lower front clip 33 are shown securing the handle of thermometer 12. Trigger 13 is shown rotatably secured to bracket 30 by way of steel shoulder screw 19.

FIG. 5C is an enlarged view of the part of the invention shown in the rectangle labeled FIG. 5C in FIG. 5A. FIG. 5C is a fragmentary front view of the foot pedal, common to both embodiments of the invention. FIG. 5C shows foot switch base 60 of pedal assembly 14 fixedly secured to conduit 15 by way of pedal EMT fitting 66.

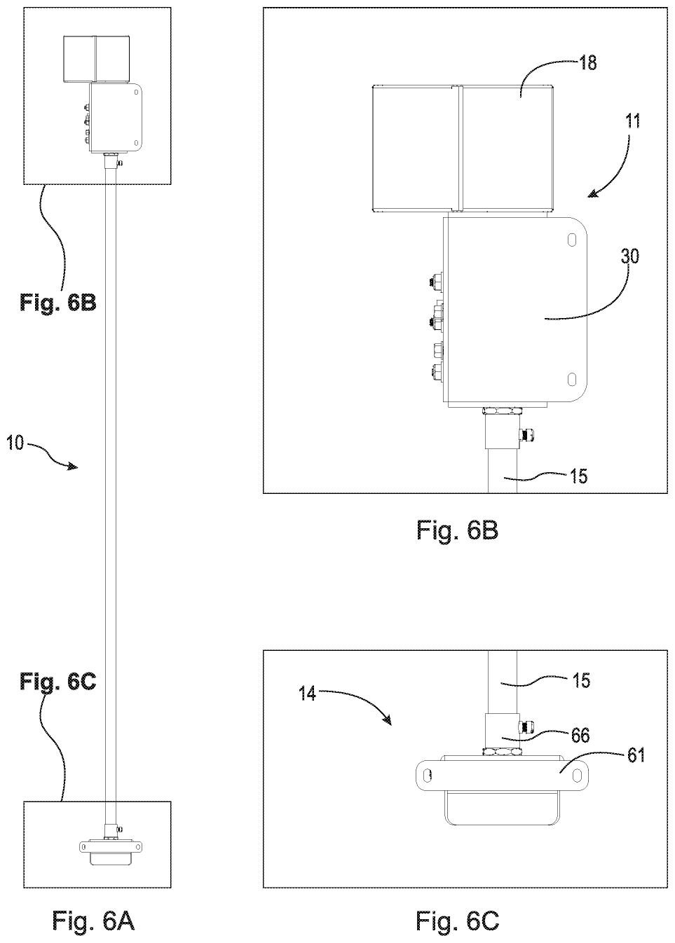

FIG. 6A is a rear view of temporal thermometer holder 10 of the first embodiment of the invention shown in FIG. 1. The following description should be read in view of FIGS. 6A-6C.

FIG. 6B is an enlarged view of the part of the invention shown in the rectangle labeled FIG. 6B in FIG. 6A. FIG. 6B is a fragmentary rear view of thermometer holding bracket and duplex mirror assembly, common to both embodiments of the invention, shown without a thermometer held in the bracket. FIG. 6B shows holder 11, bracket 30, duplex mirror 18, conduit EMT fitting 51 and conduit 15.

FIG. 6C is an enlarged view of the part of the invention shown in the rectangle labeled FIG. 6C in FIG. 6A. FIG. 6C is a fragmentary rear view of pedal assembly 14, common to both embodiments of the invention. FIG. 6C also shows conduit 15 fixedly secured to foot switch based 60 by pedal EMT fitting 66.

The following description should be read in view of FIG. 7-10. FIG. 7 is a top perspective view of the thermometer holding bracket and duplex mirror assembly, common to both embodiments of the invention, shown without a thermometer held in the bracket. Holder assembly 11 comprises duplex mirror 18 that further comprises first mirror 21, and second mirror 22, secured to bracket 30 by mirror screws 24. First mirror 21 and second mirror 22 are operatively arranged at 90° to first reflect a temperature reading from a thermometer to first mirror 21 and to reflect the mirrored image on first mirror 21 to second mirror 22 to orient the temperature reading so a user taking a temperature may record said temperature.

Bracket 30 is secured to conduit 15 by way of conduit electrical metallic tubing (EMT) fitting 51. Bracket 30 further comprises trigger 13 and trigger bar 50 that are rotatably secured to bracket 30 by way of steel shoulder screw 19. To secure a thermometer to bracket 30, upper front clip 32, lower front clip 33, upper rear clip 34, and lower rear clip 35 are operatively arranged to hold the handle of a temporal thermometer. The plurality of clips are secured to bracket 30 by a plurality of clip carriage bolts 36.

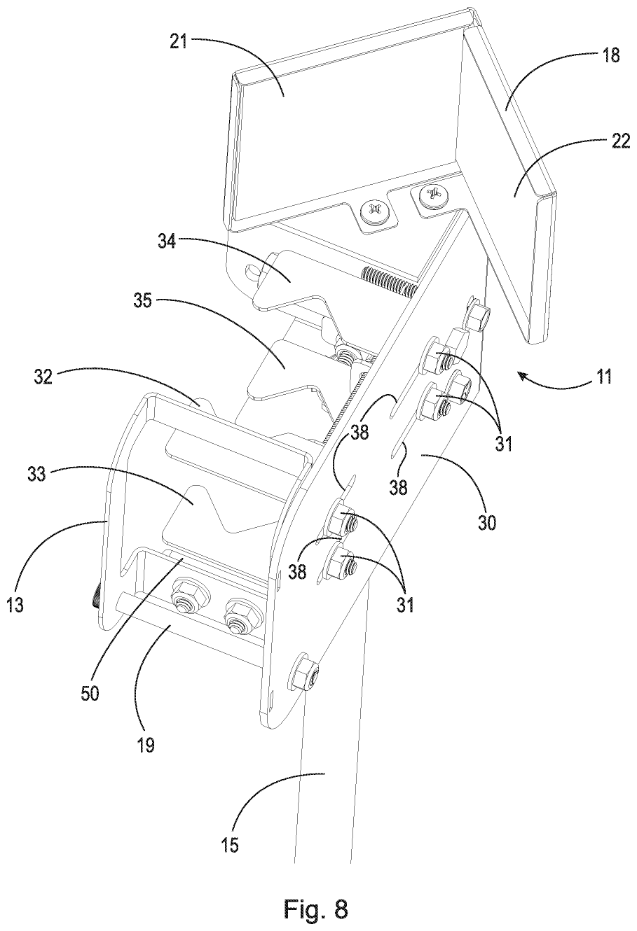

FIG. 8 is a top-right perspective view of the thermometer holding bracket and the duplex mirror assembly, common to both embodiments of the invention, shown without a thermometer in the bracket. FIG. 8 shows holder assembly 11 and conduit 15. Bracket 30 comprises, clip bolt grooves 38 that are operatively arranged so that upper front clip 32, lower front clip 33, upper rear clip 34, and lower rear clip 35 may be slidably adjustable to accommodate different sized thermometer handles. Flange lock nuts 31 secure each clip cartridge bolt 36 to maintain a desired position of thermometer clips 32, 33, 34, 35 to secure a temporal thermometer.

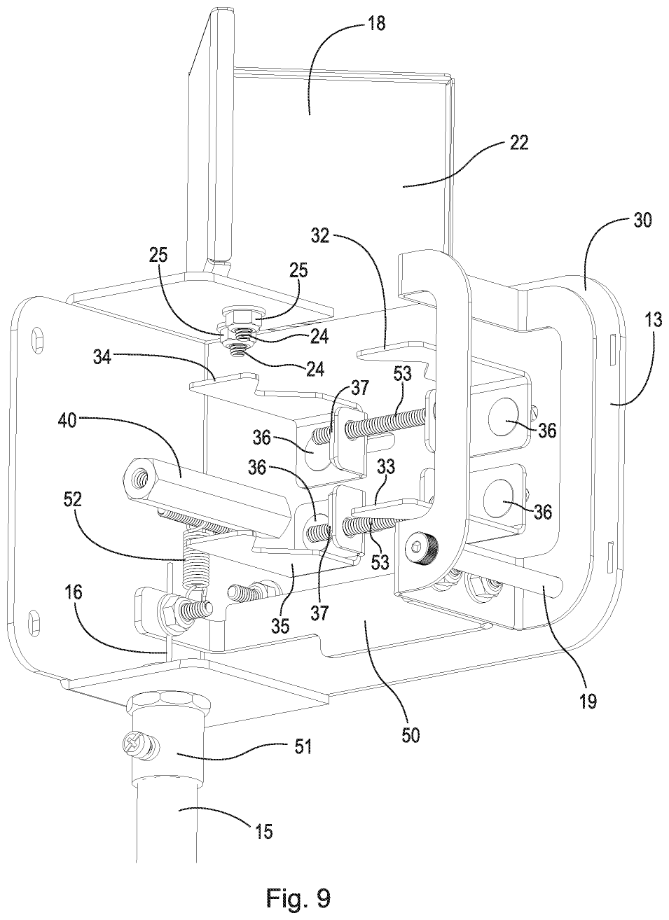

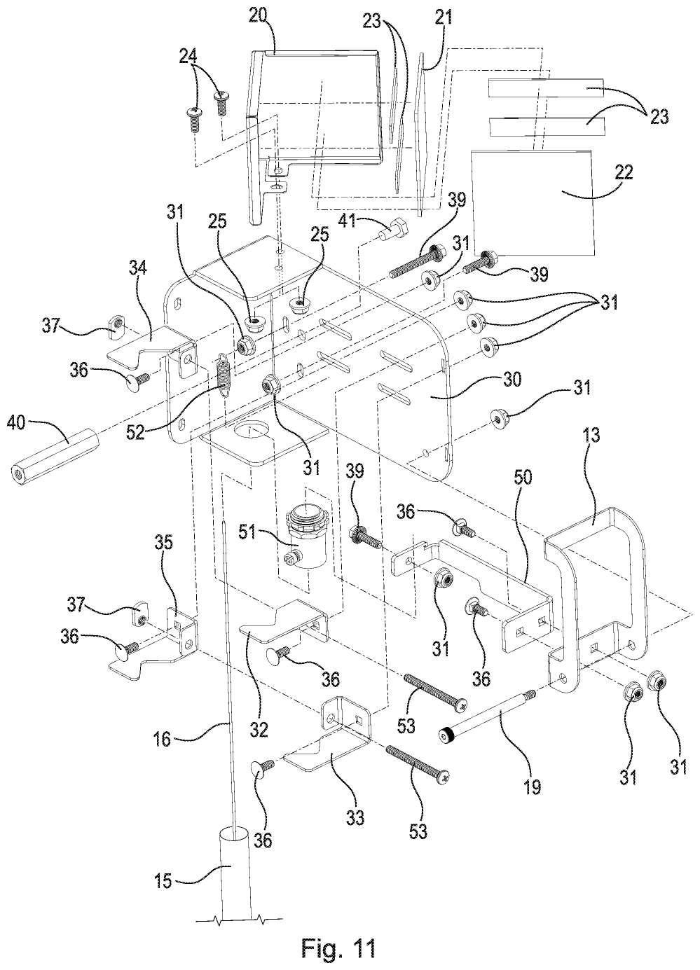

FIG. 9 is a bottom-left perspective view of the thermometer holding bracket and duplex mirror assembly, common to both embodiments of the invention, shown without a thermometer held in the bracket. FIG. 9 shows holder assembly 11 and conduit 15. Duplex mirror 18 is threadably attached to thermometer bracket 30 by way of mirror screws 24 and mirror screw flange lock nuts 25. Second mirror 22 is adhesively fixed to duplex mirror 18 by high bond tape pictured in FIG. 11, discussed infra. Upper front clip 32, lower front clip 33, upper rear clip 34, and lower rear clip 35 are shown threadably attached to clip bolt grooves 38 by way of clip carriage bolts 36 and their respective flange lock nuts 31, shown in FIG. 8 and discussed supra. Upper front clip 32 and upper rear clip 34 are fixed at a desired width by a substantially horizontal pan head machine screw 53 and clip weld nut 37. Lower front clip 33 and lower rear clip 35 are fixed at a desired width by a substantially horizontal pan head machine screw 53 and clip weld nut 37. Horizontal pan head machine screws 53 secures the respective pair of upper clips 32, 34, and the respective pair of lower clips 33, 35, such that the clips may frictionally secure or release the handle of a thermometer between said clip pairs by tightening or loosening each pan head machine screw 53 and its clip weld nut 37.

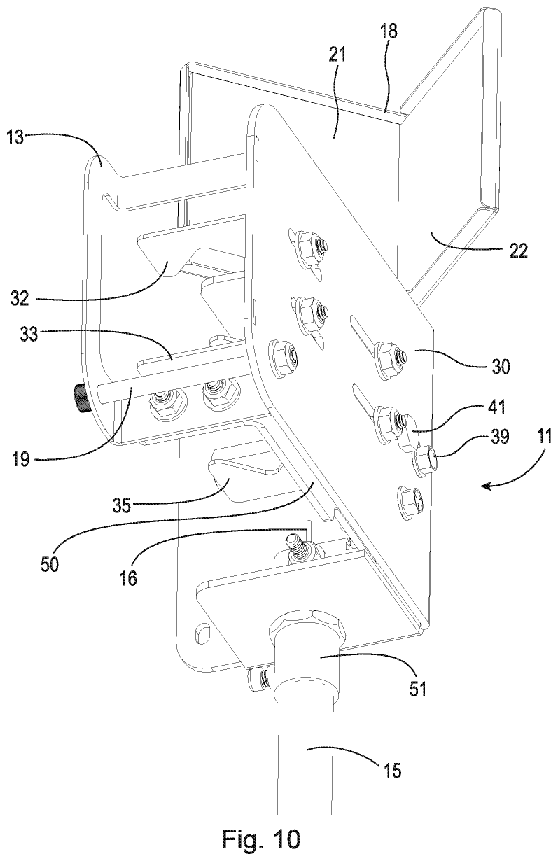

Also shown in FIG. 9 is hex standoff 40. Hex standoff 40 is threadably secured to bracket 30 by hex head bolt 41, shown in FIG. 10. Hex standoff 40 is operatively arranged to accept an optional plate or covering that would enclose the left side of bracket 30. Optional plate or covering may be threadably secured to bracket 30 by hex standoff 40.

FIG. 10 is a bottom left perspective view of the temporal thermometer holder and duplex mirror assembly without a thermometer held in the bracket. Head cap screw 39 is threadably secured to bracket 30 to secure the top loop of extension spring 52, discussed supra. FIG. 10 shows hex head bolt 41 that threadably secures hex standoff 40 to bracket 30.

FIG. 11 is an exploded view of the temporal thermometer holding and duplex mirror assembly.

FIG. 12 is an exploded view of the pedal assembly. Pedal Assembly 14 comprises foot switch base 60, rotatably secured to foot switch lever 61 by pedal shoulder screw 62. Pedal shoulder screw 62 rotatably secures foot switch base 60 to foot switch lever 61 and is threadably secured by pedal flange lock nut 63. Foot switch lever 61 contains an aperture operatively arranged to accept cable 16. Cable 16 has cable anchor 70 fixed to the distal end of cable 16 operatively arranged to prevent the distal end of cable 16 from being removed from foot switch lever 61 aperture. Pedal carriage bolt 65 is operatively arranged to pass through substantially centrally disposed apertures on foot switch base 60 and foot switch lever 61. Pedal carriage bolt 65 is threadably secured to foot switch lever 61 by a pair of pedal hex nuts 64. Conduit 15 is secured to foot switch base 60 by way of pedal EMT fitting 66.

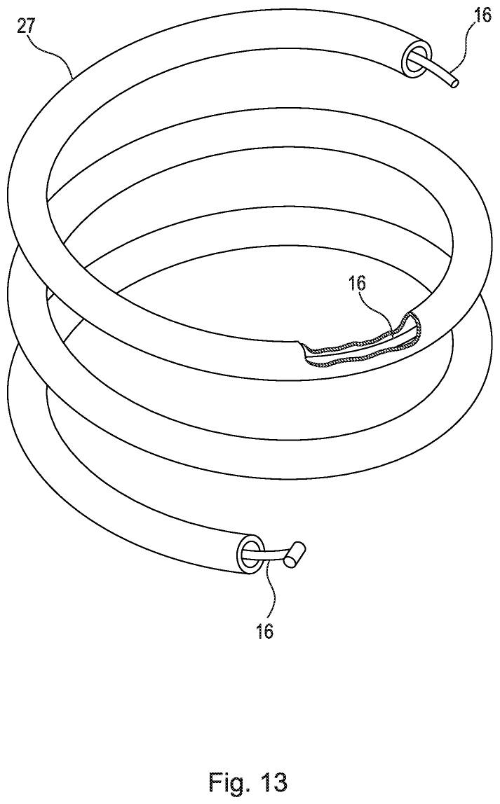

FIG. 13 is a perspective view of an alternative embodiment of the present invention's conduit fitting. An alternative conduit fitting may include flexible conduit 27 that is operatively arranged to house cable 16. Flexible conduit 27 may be used to allow for ease of shipping as it does not require specially-fit packaging to accommodate long rigid conduit 15, shown in FIGS. 1 and 3-12, described supra.

The following description should be read in view of FIGS. 1-13 and the respective description supra. It should also be appreciated that the following description applies to all embodiments of the invention.

To engage thermometer 12 to take a temperature a user first depresses pedal carriage bolt 65. Carriage bolt 65 rotates foot switch lever 61. Carriage bolt 65 is not threadably secured to foot pedal base 60 aperture and moves through the aperture when depressed, substantially functioning as a button. When foot switch lever 61 rotates, cable 16 is pulled in the downward direction from cable anchor 70 moving with foot switch lever 61.

The downward movement of foot switch lever 61 engages trigger bar 50 by the downward motion of cable 16. Trigger bar 50 and trigger 13 rotate about steel shoulder screw 19 to engage thermometer button 17 to activate thermometer 12. A user continues to depress pedal carriage bolt 65 until they have recorded the temperature that is reflected back to the user by duplex mirror assembly 18.

When a user has completed a temperature reading and releases pedal assembly 14 pedal carriage bolt 65, extension spring 52 pulls cable 16 in a vertical direction to maintain a substantially level position of trigger bar 50 that releases trigger 13 from depressing thermometer button 17.

### LIST OF REFERENCE NUMBERS

10—temporal thermometer holder
11—holder assembly
12—thermometer
13—trigger
14—pedal assembly
15—conduit
16—cable
17—thermometer button
18—duplex mirror
19—steel shoulder screw
20—mirror bracket
21—first mirror
22—second mirror
23—high bond tape
24—mirror screw
25—mirror screw flange lock nut
27—flexible conduit
30—bracket
31—flange lock nut
32—upper front clip
33—lower front clip
34—upper rear clip
35—lower rear clip
36—clip carriage bolt
37—clip weld nut
38—clip bolt grooves
39—head cap screw
40—hex stand off
41—hex head bolt
50—trigger bar
51—conduit electrical metallic tubing (EMT) fitting
52—extension spring
53—pan head machine screw
60—foot switch base
61—foot switch lever
62—pedal shoulder screw
63—pedal flange lock nut
64—pedal hex nut
65—pedal carriage bolt
66—pedal electrical metallic tubing (EMT) fitting
70—cable anchor

What I claim is:

1. A holder for a temporal thermometer, comprising:
a bracket arranged to hold said thermometer;
a lever operatively arranged to depress a button on said thermometer to take a temperature reading;
a foot pedal;
a cable linking said foot pedal to said lever; and,
a duplex mirror assembly operatively arranged to display said temperature reading.

2. The holder recited in claim 1 further comprising a conduit arranged to cover and surround said cable.

3. The holder recited in claim 1 wherein said duplex mirror comprises a first mirror and a second mirror, and said first and second mirror are arranged at a substantially right angle relative to one another.

4. The holder recited in claim 3 wherein said thermometer includes a display, and said first mirror is arranged at substantially a 45 degree angle relative to said display.

5. The holder recited in claim 1 wherein said bracket includes a threadably secured outer cover.

6. The holder recited in claim 1 further comprising a flexible conduit arranged to cover and surround said cable.
